(19) United States
(12) Patent Application Publication
(10) Pub. No.: US 2010/0279248 A1
(43) Pub. Date: Nov. 4, 2010

(54) DEVICE AND METHOD FOR PREDICTING THE LIKELIHOOD OF CARIES DEVELOPMENT

(76) Inventors: Pierre D. Mourad, Seattle, WA (US); Joel Berg, Bellevue, WA (US); Paul Ray Illian, Seattle, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 12/718,879

(22) Filed: Mar. 5, 2010

Related U.S. Application Data
(60) Provisional application No. 61/157,857, filed on Mar. 5, 2009.

(51) Int. Cl. A61B 6/14; G06N 5/02

(52) U.S. Cl. 433/29; 706/52

(57) ABSTRACT

A hand-held intra-oral dental device and method are described for the detection of pre-caries lesions and the prediction of evolution and prognosis of same. The present invention has as its foundation a low-cost tool for predicting the likelihood of the development of Early Childhood Caries (ECC), in contrast to other techniques and associated devices, where their focus is to identify individual pre-caries lesions. This method focuses on the detection of caries precursors or of their patterns and the relationship of those precursors and patterns to the likelihood of subsequent dental disease. The implications for the establishment for early preventive treatment are profound, namely the earliest implementation of preventative therapy for our approach relative to that of the other approaches.

### RELATED APPLICATIONS

This application claims the benefit of U.S. Provisional Application No. 61/157,857, filed Mar. 5, 2009, the disclosure of which is hereby expressly incorporated by reference in its entirety.

### FIELD OF THE INVENTION

This invention relates to detection of at least a subset of pre-caries lesions within a given patient's mouth for purpose of predicting the likelihood that that patient will develop caries lesions somewhere in their mouth in the near future, as a means of motivating targeted therapy that will prevent the dental caries from actually occurring.

### BACKGROUND OF THE INVENTION

Dental caries, otherwise known as cavities or tooth decay, are lesions in the enamel and underlying dentin in teeth. Treatment consists of removing the damaged dentin and enamel with a drill and filling the resulting divot with one of a variety of materials. Besides the significant physical and emotional cost of cavities, there is also a significant cost of dental care once a cavity forms, which is currently estimated at approximately US $3500 over the lifetime of an individual per cavity. This sum covers the treatment of the cavity, possible re-treatment of the cavity, possible root canal, dental bridges, etc. Currently, between US $7-$14 billion is spent annually in the United States annually for dental care, with this market growing at an annual rate of 7%. In the United States and the world as a whole, the urban poor and rural populations suffer disproportionately from this disease due to financial and logistical hardship.

The surface and near surface of teeth (enamel, dentin, cementum) are in a constant state of de-mineralization and re-mineralization (J. D. Featherstone, “The continuum of dental caries-evidence for a dynamic disease process”, J. Dent. Res., 2004 83 Spec No C: C39-42). For example, the acid generated by the action of plaque bacteria acting on and just below the surface of teeth de-mineralizes the enamel. This demineralization process is balanced, typically, through re-mineralization facilitated by the presence of fluoride, calcium and other products within saliva that both reduce the low pH created by the plaque bacteria and create new crystalline material. However, the further along a defect progresses, the more likely that demineralization will continue. Eventually, the affected enamel, for example, will collapse, or cavitate (hence the term “cavity”), due to the action of plaque bacteria on the enamel. The cavity exposes the underlying dentin, which is vulnerable to further attack and decay. If altered enamel can be identified before the dentin is exposed, topical application of a fluoride varnish every six weeks for six months, along with best oral health practices, can build normal enamel back to healthy levels, thereby preventing the creation of a cavity.

This preventative process is particularly critical for young children, who typically experience their first visit to a dentist at the age of three or four years. Regrettably, such a regimen of preventative treatment is sufficiently inconvenient and expensive (primarily in terms of time and logistics) that every child cannot receive such therapy on a regular basis to automatically help prevent caries development. Children begin their lives free of dental disease as their primary dentition erupts between the ages of 9 and 18 months. However, early childhood caries has been observed to devastate dentition sufficiently to motivate surgical intervention in 10-15% of young children, with a greater number experiencing otherwise adversely impacted overall health. Early childhood caries are generally not clinically manifested and detectable in children at one year of age, but often manifested in a significant way by ages 2-2½ years.

Fortunately, “pre-caries lesions”, or dental caries in their earliest stages, can be treated to arrest further caries development or even fix the crystalline defect by the fluoride regimen previously described. Thus, there is a tremendous opportunity to intervene and prevent the occurrence of the disease in a large portion of the population of young children if one could identify the children at greatest risk at the earliest possible stage. For example, the American Association of Pediatricians (AAP) recommends 10-12 well-baby check-ups prior to the third birthday, wherein an opportunity exists to screen infants and toddlers for pre-caries activity that is not yet clinically evident, provided suitable screening tools are available. As a general rule, pediatricians see children under the age of four or five far more often and regularly than do dentists, even those trained as pediatric dentists. Ideally, a pre-caries detection tool in the hands of a pediatrician could, during the course of a regular check-up, fulfill the function of screening for pre-caries lesions, determine whether or not early treatment is warranted, and allow the pediatrician to advise parents to have the child seen early on by a dentist for further examination and preventative treatment.

Loss of mineral from tooth enamel and dentin has been known since the 1960s to change the optical properties of teeth (G. K. Stookey, “Optical methods-quantitative light fluorescence”, J. Dent. Res., 2004 83 Sec No. C: C84-8). Documentation of technological development of caries-detecting devices based on optical sensing has appeared in the patent and non-patent literature since the early 1980s. Early examples of such development are documented in U.S. Pat. No. 4,290,433 (Alfano), which describes a method and apparatus for detecting the presence of caries in human teeth using differences in visible luminescence spectra of decayed and healthy enamel. U.S. Pat No. 4,479,499 (Alfano) discloses a method and apparatus for detection of dental caries wherein the tooth is illuminated by two wavelengths of visible light, and detection of caries is accomplished when the difference in the intensity of the light scattered from the tooth monitored at the chosen wavelengths changes in a predetermined manner.

Natural auto-fluorescence of teeth was found to occur when it was observed that the use of laser fluorescence enhanced the contrast between areas of demineralization and sound enamel (H. Bjelkhagen et al., “Early detection of enamel caries by the luminescence excited by visible laser light”, Swed. Dent. 1982 6: 1-7). U.S. Pat. No. 4,515,476 to Bjelkhagen also discloses a device to evaluate teeth using a visible luminescence visual signal. It was later reported that laser fluorescence could quantitatively assess enamel demineralization in vitro that compared well with microradiography for the measurement of mineral changes (U. Hafstrom-Bjorkman et al., “Comparison of laser fluorescence and longitudinal microradiography for quantitative assessment of in vitro enamel caries”, Caries Res. 1992 26: 241-7). Since that time, numerous studies have been conducted to document fluorescent changes with demineralization and remineralization of the enamel surface (M. Ando et al., “Comparative study to quantify demineralized enamel in deciduous and permanent teeth using laser- and light-induced fluorescence techniques”, Caries Res. 2001 35: 464-70; H. Eggertsson et al., “Detection of early interproximal caries in vitro using laser fluorescence, dye-enhanced laser fluorescence and direct visual examination”, Caries Res. 1999 33: 227-33); M. D. Lagerweij et al., “The validity and repeatability of three light-induced fluorescence systems: An in vitro study”, Caries Res. 1999 33: 220-6; X. Q. Shi et al., “Comparison of QLF and DIANGOdent for quantification of smooth surface caries”, Caries Res. 2001 35:21-6; S. Tranaeus et al., In vivo repeatability and reproducibility of the quantitative light-induced fluorescence method“, Caries Res. 2002 36: 3-9). Research has indicated a good correlation between the change in average fluorescence radiance and the average change in mineral content of the tooth surface as measured with transverse microradiography (M. Ando et al., Relative ability of laser fluorescence techniques to quantitate early mineral loss in vitro”, Caries Res. 1997 31: 125-31; Eggertsson et al. 1999 (citation supra), Z. Emami et al., “Mineral loss in incipient caries lesions quantified with laser fluorescence and longitudinal microradiography. A methodologic study” Acta Odontol. Scand. 1996 54: 8-13). Additional studies correlated the change in the depth of caries lesions with the average change in the fluorescence radiance in permanent teeth (Ando et al. 1997 (citation supra); A. F. Hall et al. , “In vitro studies of laser fluorescence for detection and quantification of mineral loss from dental caries.” Adv. Dent. Res. 1997 11: 507-14) as well as children's teeth (M. Ando et al., “Comparative study to quantify demineralized enamel in deciduous and permanent teeth using laser- and light-induced fluorescence techniques.”, Caries Res. 2001 35: 464-70). Much of the in vitro caries research has investigated lesion sizes averaging 0-50 μm in depth, with an associated 10-15% corresponding change in fluorescence.

Several devices employing fluorescence-based detection methods have been recently commercialized. The general method has been termed quantitative light-induced fluorescence (QLF), and operates on the principle that sound, healthy tooth enamel yields a higher intensity of fluorescence under excitation from high intensity blue light than does de-mineralized enamel that has been damaged by caries infection. The high degree of correlation between mineral loss and loss of fluorescence for blue light excitation is then used to identify and assess carious areas of the tooth. A different relationship has been found for red light induced fluorescence, a region of the spectrum for which bacteria and bacterial by-products in carious regions absorb and fluoresce more pronouncedly than do healthy areas.

The current state-of-the-art is replete with examples of hand-held or easily deployable devices and methods for clinical detection of pre-caries lesions and dental caries using QLF techniques. U.S. Application 2006/0240377 (De Josselin et al.) discloses a hand implement for inspecting and detection of abnormal tooth surface conditions, and method for using same. The device uses QLF to distinguish abnormal enamel surface, including plaque-covered enamel, from healthy enamel by visually observable differential fluorescence signals, where the differential signals are visible to a trained practitioner performing the inspection via an attached mirror for immediate detection of sites of dental caries or other pathologies. This patent has been commercialized under the brand QLF™ In Vitro (www.inspektor.nl), a device whose primary target is the identification of caries, though it has also been used in research to track the remineralization of teeth (S. Tranaeus et al., “Application of quantitative light-induced fluorescence to monitor incipient lesions in caries-active children: A comparative study of demineralization by fluoride varnish and professional cleaning” Eur. J. Oral Sci. 2001 109: 71-75).

U.S. Pat No. 6,102,704 (Elbofner et al.), which has been commercialized under the name DIAGNODent™ by KaVo (www.kavousa.com), U.S. Application No. 2008/0102416 (Karazivan), which has been commercialized under the name Midwest Caries I.D.™ by Dentsply Canda Ltd. (www.cariesid.com), U.S. Pat. No. 6,561,802 (Hack) and U.S. Pat. No 7,270,543 (Stookey et al.) all disclose a hand held fluorescence probe resembling a standard dental instrument for detection of caries and other dental pathologies, whereby the probe uses a LED light source to stimulate local QLF, measures and evaluates same and displays the result to the user on a readout to the user. U.S. Pat. No. 6,102,704. This device has been shown to be insufficiently specific for caries detection, highlighting all kinds of defects in a very sensitive way that are not necessarily cavities (pre-caries lesions for example), leading therefore to more drilling and filling than is warranted (J. D. Bader and D. A. Shugars, “A systematic review of the performance of a laser fluorescence device for detecting caries” J Am Dent Assoc. 2004 135: 1413-26)

U.S. Pat. No. 7,577,284 (Wong et al.) discloses a CCD-based imaging device using a combined QLF image and visible light image of a tooth to provide a high contrast image for enhanced caries visualization on tooth surfaces.

U.S. Application 2008/0118886 (Liang et al.) discloses the combination of optical coherence tomography (OCT) imaging, also used in medical imaging applications, to provide very detailed imaging of structure beneath the surface of a tooth, including the depth penetration of the caries into the tooth, combined with white light or fluorescence imaging of the tooth in order to 1) pinpoint localized areas of interest by localizing particular teeth that appear prone to pre-carious lesions, and 2) perform OCT scans in this areas of interest on the tooth to clearly image details of regions of tooth enamel undergoing early demineralization in contrast to non-diseased enamel.

As an example of a non-fluorescent technique, U.S. Pat. No. 6,522,407 (Everett et al.) discloses the method of illuminating dental tissue with polarized light and measuring the polarization state of the backscattered light. Changes in the polarization state are caused by demineralization of the enamel. A hand-held fiber optic dental probe is used in vivo to direct the incident beam to the dental tissue and collect the reflected light. In another example of devices and methods, U.S. Application No. 2007/0134615 (Lovely) discloses a dental imaging system that uses near infrared light between 800 and 1800 nm either transmitted through or scattered from the tooth under examination.

### SUMMARY

While the technology of pre-caries detection has advanced to a high degree, these technologies are focused, primarily, in their commercial manifestations on detection of existing dental caries alerting dental professionals to the presence of a fully developed caries lesion requiring invasive drill and fill treatment. As noted, however, these devices can detect pre-caries lesions, and have even been proposed as useful for tracking pre-caries lesions. However, given the nearly continual demineralization and remineralization activity on and within the surface of teeth and the ability of targeted intervention to support remineralization, there is still a great need for a commercial device and method to not only detect early onset of carious lesions (‘pre-caries’ lesions) but also to use that information to predict the likelihood of subsequent cavity formation for the purposes of targeting early treatment and prevention to those patients at greatest risk of subsequent cavity formation.

The present invention addresses this long-felt need. In some embodiments, the invention includes methods, devices, and systems that are configured to detect pre-carious lesions and to predict the prognosis of such lesions. In additional or alternative embodiments, the invention can also include detection and prediction of other lesions located elsewhere in the mouth and having a history that correlates with that of those observed or is otherwise predictable from those observed. In some embodiments those predictions will be based strictly on the detectable characteristics of the lesions interrogated by the device or correlated with those lesions interrogated by the device. In addition, or in a complementary way, epidemiological studies may produce clinically prognostic relationships between detectable characteristics of the lesions within a given patient's mouth and the clinical outcome of a separate cohort of patients with comparable lesion characteristics. In general, embodiments of the invention incorporate conventional radiative techniques, such as those described above, or other conventional techniques, including fluoroscopy methods, optical coherence tomography (OCT), Raman spectroscopy, near infrared illumination in transmission or backscatter mode, and ultrasound. However, unlike conventional radiative techniques or other techniques, embodiments of the present invention incorporate a predictive algorithm that is based on the comparison of measurement data and epidemiological data. Also, embodiments of the invention provide increased specificity for pre-caries lesions relative to actual cavities. It should be clear to those versed in the state of the art that the other detection methods sketched here can be used to detect pre-caries lesions sufficiently well to predict the likelihood of pre-caries lesions evolving into cavities, either at the site of detection, or in other areas within the mouth that have a comparable propensity to evolve into caries lesions, likely because of their comparable history of demineralization and remineralization.

In some embodiments, the invention comprises an electronic signal processing system designed to analyze the backscattered or transluminescence signals emanating from the tooth under examination as a result of using any one of the methods described above on one or more teeth of a patient. The signal processing system includes a microprocessor, an execution code storage memory device such as an electronically erasable programmable read only memory (EEPROM), random access memory (RAM) and is by no means restricted to, image processing by pixel analyses based on central tendency, measures about a fixed or central measure, measures of the percentage of pixels above a set threshold with a region of interest, and measures of spatial distribution about a peak pixel value. The signal processing system can further include single point analysis, which portends the assay of the integrated effect of the presence of pre-caries on the propagation through or scatter of incident energy from the tooth surface, as anticipated, for instance, by the integrated detection over a region of interest of the tooth under examination of optical signals such as back-scattered luminescence, transluminescence, or by ultrasound surface acoustic wave analysis or analysis of echo signals resulting from longitudinal ultrasound impulses.

In some embodiments, a predictive algorithm provides for an evaluation of the likelihood of cavity formation anywhere in a given patient's mouth for the purposes of targeting early treatment and prevention to those patients at greatest risk of subsequent cavity formation, wherein data resulting from the signal processing means are subsequently analyzed by the predictive algorithm means. The algorithm comprises the steps of extracting from the image scatter or propagation data one or more of a variety of geometric factors characteristic of the subset of the entire patient's pre-caries lesion population that have contributed to the data. These geometric parameters can include, for example, the mean, median or mode of the surface area of the pre-caries lesions weighted by the brightness or darkness of those lesions relative to the mean, median or mode of the level of reflectivity, emissivity, attenuation, etc., of the entire tooth. These geometric parameters are then correlated with epidemiological data obtained from studies on patient peer populations in order to assign probabilities relating to the likelihood of evolution of the pre-caries lesions into full caries over a specified time period.

In some embodiments, a hand held dental examination instrument includes a handle means is affixed to a head portion means for intra-oral examination, the head portion means comprising a radiant energy source capable of irradiating a portion of a tooth under examination and inducing a back-scattered signal emanating from the surface and/or near surface region of the tooth enamel, or a transluminescence signal propagated through, the irradiated tooth; a detector means simultaneously disposed within the head portion to capture the back-scattered or transluminescence (propagated) signals. The instrument further comprises a microprocessor means, an electronic memory means and associated electronic circuitry, a visual signaling system, and a data link interface to export data to a remote computer for real time data monitoring, such as imaging, displaying of examination results, or uploading of measurement data.

### BRIEF DESCRIPTION OF THE DRAWINGS

FIG. 1a is a side view of a hand-held instrument showing cross-sectional overall detail of a hand-held embodiment of the instrument containing LED indicator visual display.

FIG. 1b is a side view of a hand-held instrument showing cross-sectional overall detail of a hand-held embodiment of the instrument containing a numerical readout display.

FIG. 2 shows detail of the optical configuration of the probe head.

FIG. 3 depicts a flow chart describing a predictive algorithm implementation.

FIG. 4 depicts a block diagram showing the functional relationship between the electronic processing subsystem and the other subsystems, including the predictive algorithm.

FIG. 5a shows a fluorescent image of a tooth with a known cavity (dark spot) surrounded and adjacent to know naturally occurring pre-caries lesions.

FIG. 5b shows the delineation of a region of interest (ROI) of the same tooth of FIG. 5a.

FIG. 6a depicts a contour plot of a region of interest (ROI) of FIG. 5b.

FIG. 6b shows a line scan of a cross section of the ROI in a lesion-free section at one side of ROI.

FIG. 6c shows a line scan of a cross section of the ROI containing a caries and pre-caries lesion.

FIG. 6d shows a line scan of a cross section of the ROI in a lesion-free section at a side opposite that of FIG. 6b.

FIG. 6e shows a grayscale image of the ROI where degree of darkness is correlated to enamel demineralization and ranges in darkness as normal (white), precaries (shades of grey) and cavity (black).

### DETAILED DESCRIPTION

Pre-carious lesions are demineralized volumes at and near the surface of a tooth, typically in the enamel, that have varying degrees of depth, while the underlying dentin is not yet affected by the cariogenic attack. In the most advanced cases, the enamel is completely penetrated and underlying dentin is exposed to attack. For early childhood caries prevention programs, it is desirable to have the ability to predict the occurrence of caries development based on detection of pre-caries lesions. As described above, this can be particularly true for young children. It is known that pre-caries lesions can exist in one of two states or phases, 1) a progressive demineralization phase, and 2) a progressive natural remineralization, or healing phase. The latter may occur when cariogenic conditions disappear in the mouth, and natural rebuilding of the injured enamel can occur by re-deposition of calcium phosphate carried in the saliva (B. T. Amaechi and S. M. Higham, “In vitro remineralization of eroded enamel lesions by saliva”, J. Dent. 2001 29: 371-6; J. D. Featherstone, “The continuum of dental caries-evidence for a dynamic disease process”, J. Dent. Res., 2004 83 Spec No C: C39-42). It is important to distinguish between these two states, or to infer the existence of pre-caries lesions in places in the mouth where they are hard to detect, in order that when a given population of pre-caries lesion is found, and assayed, its tendency towards further remineralization or demineralization or the tendency of pre-caries lesions elsewhere in the mouth to remineralize or demineralize can be known for indicating the overall dental health of the patient, and whether or not a course of treatment is indicated, and if so, how soon it should be undertaken. Epidemiological studies on the progression of caries in young children exist, (A. P. Vanderas et al., “Progression of proximal caries in children with different caries indices: a 4-year radiographic study”, Eur Arch Paediatr Dent. 2006 7: 148-52) and have been shown to be useful as an overall determiner of the prognosis of development of carious lesions in surrounding sound enamel of the same tooth and nearby teeth of an individual, in addition to predicting the rapidity of decay of the enamel at the site of the existing lesion.

Embodiments of the present invention combine the predictive power of epidemiological data for prognosis of pre-carious lesions, described above, with technological advances in the in-vivo detection of pre-carious lesions for the purpose of early detection and prevention of caries in children. To this end, the present invention comprises a device that measures regions of the tooth surface, typically the enamel, identifying a pre-carious lesion or a population of pre-caries lesions, and an algorithm for processing the digital information of the pre-caries lesion and predicting its prognosis as well as comparable prognosis for the other teeth in the mouth.

In some embodiments, a portable instrument can further provide for ease of handling by non-dental practitioners, such as pediatricians (pediatric physicians), nurses and other medical personnel, as well as trained dental professionals, to routinely perform cursory pre-caries screenings on children in the course of general health check-ups in a clinical or field setting. Embodiments of the instrument include data processing capabilities, including the implementation of a predictive algorithm to determine the prognosis of specific pre-carious lesions or populations of pre-caries lesions as well as the prognosis of ancillary populations of pre-caries lesions in the same mouth but not directly detected by the device, and to display the results of prognosis in a simple visual signaling manner to the practitioner-user of the instrument who is performing the examination, in addition to being capable of uploading image data and predictive results to a remote computer by wired or wireless data link. The practitioner-user can then advise the child's parent and/or dentist on the apparent severity of the situation, whereby the latter can further evaluate the child's condition.

In some embodiments, a portable, hand-held instrument includes a probe head and handle that can be positioned in the mouth of a child in a fashion similar to that with which one would hold typical dental examination instruments.

In another embodiment, an electronic processing system includes a central microprocessor unit and one or more memory storage devices, such as erasable programmable read-only memories (EPROM), flash memory, and/or other volatile or non-volatile memories. The memory storage devices can include computer executable code for a variety of functions, including controlling a source of radiant energy, controlling a detector, signal processing and for executing the steps of the predictive algorithm.

In another embodiment, a visual signaling system is included to indicate to the user-practitioner the result of the analysis performed by the instrument. One example of a visual signaling system is a series of colored LEDs disposed on the body of the instrument, wherein each color represents a range of probabilities that a detected pre-carious lesion will develop into a dental caries within a set time period, such as an 6-18 month period indicated by example. By example, a practitioner-user would be alerted to the presence of a detected (pre)carious lesion by illumination of one of the LEDs, which, by one of the colors, red, yellow, or green, would in turn immediately indicate to the practitioner-user the level of probability by which the lesion or other lesions in the mouth will develop into a dental caries (cavity). The practitioner-user can take further action based on the indication, where, by example, the green LED remains illuminated as long as 1) no pre-caries lesions are detected, or possibly 2) a pre-caries lesions are detected but determined likely to be in a re-mineralization phase (auto-healing phase) and have a very low probability of developing into a cavity, whereas the red LED illuminates when a lesion is detected and determined to have a large degree of demineralization and immanent or actual penetration into the dentin, indicating a very high probability of developing into a cavity, or may already be a cavity, both cases requiring immediate post-examination action to treat the lesion, as an example. The yellow LED illuminates when a pre-carious lesion is detected, wherein the degree of deminerization is indicative of an intermediate probability of developing into a lesion penetrating into the dentin within a 6-18 month time frame, not requiring immediate post-examination treatment, but should be followed up either with a preventative treatment such as a series of fluoride varnish applications and targeted education, or be monitored periodically by a dental professional to follow the progress of the lesion. As an important alternative, all pre-caries lesions may be associated with the yellow light, reserving the green light for the case of a low likelihood that any pre-caries lesions exist anywhere on the dentition and the red light for a high likelihood that an actual cavity exists somewhere in the dentition. What range of disease state is associated with what color depends significantly on the best clinical practice for each circumstance.

A further example of a visual signaling system is a numerical readout, wherein a digital numerical indicator or dial is positioned on the device for a numerical indication of the degree of severity of detected lesions. Numerical values are assigned to probabilities or ranges of probabilities, indicating to the practitioner-user the likely subsequent degree of progress of the detection lesion, similar to the color signals described above. As a further example, a visual signaling system is a display of results on a monitor screen, either integral with the device or on a separate laptop or desktop computer monitor screen.

In another embodiment, a data communications link is provided on the instrument to allow wired or wireless connection with a computing device, such as a laptop computer or hand-held mobile device, for visualization of tooth image “raw” data in real time. Examination results can be uploaded to the connected computer for storage and printout in hardcopy form.

Generally, embodiments of the invention employ epidemiological data specific to the patient (age, race, gender, their diet and that of their care giver, dental history, etc) in addition to the technologically derived data discussed above, which can include a separate cohort of patient's with known clinical outcome to make that technology useful in order to make a prediction of the risk that that patient has for developing caries.

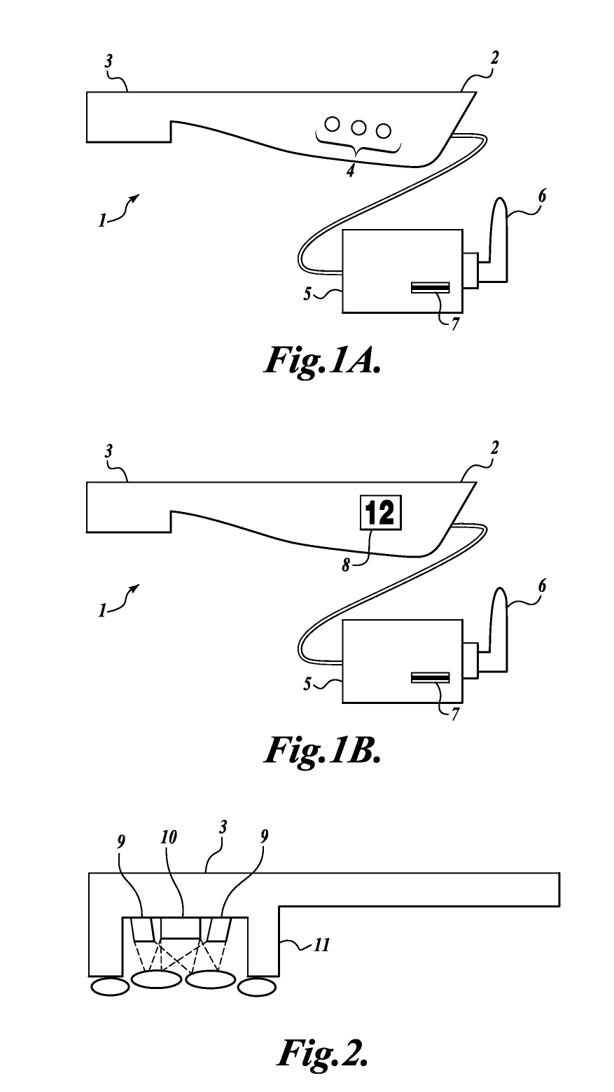

Referring to FIG. 1a, the instrument 1 comprises a handle portion 2 and a probe head 3. A plurality of luminous colored indicators 4, comprising a visual signaling system, are shown disposed along the handle portion 2, but by no means are limited to this particular arrangement. In other embodiments, the luminous indicators 4 can be disposed anywhere along the handle portion 2 or probe head 3, or on the housing of interface 5. Interface 5, equipped with a wireless antenna 6 or USB 2.0 connector 7 (or any other suitable data communications connector such as a RS 232), can be used for a wireless or wired data link, respectively, with an external computer for real time data imaging and for uploading examination results including prognosis results. In a preferred embodiment, the colored indicators 4 are LEDs. Each color of the LEDs 4 represents a range of probabilities that a detected pre-carious lesion will develop into a dental caries within a set time period, such as an 6-18 month period indicated by example. By example, a practitioner-user would be alerted to the presence of a detected carious lesion by illumination of one of the LEDs 4, which, by one of the colors, red, yellow, or green, would in turn immediately indicate to the practitioner-user the level of probability by which the lesion will develop into a dental caries (cavity). The practitioner-user can take further action based on the indication, where, by example, the green LED remains illuminated as long as 1) no pre-caries lesions are detected, or, possibly 2) pre-caries lesions are detected but determined likely to be in a re-mineralization phase (auto-healing phase) and have a very low probability of developing into a cavity, whereas the red LED illuminates when a lesion is detected and determined to have a large degree of demineralization and immanent penetration into the dentin, indicating a very high probability of developing into a cavity, or may already be a cavity, both cases requiring immediate post-examination action to treat the lesion, as an example. The yellow LED illuminates when a pre-carious lesion is detected, wherein the degree of deminerization is indicative of an intermediate probability of developing into a lesion penetrating into the dentin within a 6-18 month time frame, not requiring immediate post-examination treatment, but should be followed up either with a preventative treatment such as a series of fluoride varnish applications and targeted education, or be monitored periodically by a dental professional to follow the progress of the lesion. As an important alternative, all pre-caries lesions may be associated with the yellow light, reserving the green light for the case of a low likelihood that any pre-caries lesions exist anywhere on the dentition and the red light for a high likelihood that an actual cavity exists somewhere in the dentition. What range of disease state is associated with what color depends significantly on the best clinical practice for each circumstance

In another embodiment shown in FIG. 1b, the visual signaling system comprises a numerical readout 8 disposed along the handle portion 2. The numerical readout 8 is by no means confined to this particular arrangement. In other embodiments, numerical readout 8 can be disposed at any point along the handle portion 3 or probe head 2, and furthermore can be disposed on the housing of interface 5. Numerical values are assigned to probabilities or ranges of probabilities, indicating to the practitioner-user the degree of progress of the detection lesion, similar to the color signals described above.

In one embodiment, quantified light-induced fluorescence (QLF) is employed as the method of tooth enamel analysis. Irradiation is implemented with near infrared, blue or ultraviolet light. The source can be a fixed array of LEDs or a scanning optical fiber or minor. Autofluorescence capture is accomplished with a detector in the form of imaging optics integrated with a CCD camera capable of transducing portions of the auto-fluorescence spectra emanating from the tooth enamel into electronic signals in the form of a one or two dimensional pixel-based image. The present embodiment further comprises a microprocessor for controlling the illumination source and CCD camera detector, as well as for processing the acquired pixel images by as a first step recognizing the presence of one or more pre-carious lesions contained within the image, this done by analyzing the pattern of fluorescence intensity values contained in each pixel, and as a second step subjecting the acquired pixel image to a predictive algorithm in order to assign a probability to the pre-carious lesions detected in the image of developing into dental caries within a set time period. The output of the algorithm is conveyed to a practitioner-user by a visual signaling system in real time, or by generation on a remote device of a summary of the examination to be read subsequently.

In other embodiments of the invention, the detector can comprise, but by no means be limited to, a single photodiode, a photodiode array or an array of one or more thermopile elements for non-image data.

Referring now to FIG. 2, details of probe head 3 are described for incorporation of an integrated QLF system. Probe head 3 comprises a plurality of LED illumination sources 9 capable of exciting auto-fluorescence of the enamel, a CCD intra-oral camera 10 complete with integrated optics, capable of receiving and recording light energy in the spectral range of the auto-fluorescence, and a standoff 11 to maintain proper focal distance when positioned against the patient's teeth, as shown in the figure. In one embodiment, the LED sources 9 can have a spectral output centered for example around 405 nm as yielding optimum fluorescence based on published studies. LEDs 9 can be disposed in several configurations in corresponding embodiments, including, but by no means limited to, a single LED, dual LEDs, quadruplet LEDs, and also in a series of concentric rings. In a preferred embodiment, the specifications of the CCD chip and integrated optics of the intra-oral camera 10 are similar to those used in the Magenta Technology Co. MD-750 intra-oral camera, having a pixel resolution of 1280×960, and a focal length of 10 mm.

In some embodiments of the invention, the predictive algorithm can be implemented as firmware in an EPROM or an EEPROM chip, which can be integrated within the body of the hand-held instrument in one preferred embodiment or can be disposed in the interface 5 in another embodiment.

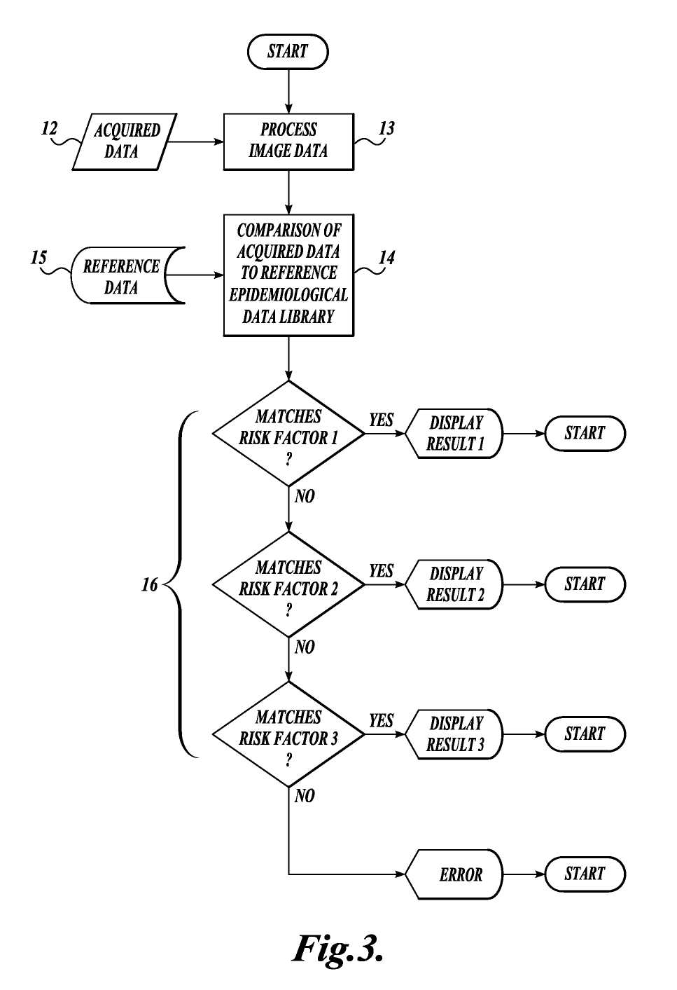

One embodiment of the predictive algorithm is described by the flowchart in FIG. 3. Acquired raw data 12 in the form of an image pixel array are processed by program module 13 and to obtain a value. The subroutine used in module 13 comprises, and is by no means limited to, 1) measurement of a central tendency within the region of interest to quantify the degree of damage to the enamel within the region of interest of an examined tooth, 2) measurement of the percentage of pixels values (or their derivatives, or variance, etc.) above a set threshold within the region of interest to quantify the degree of damage to the enamel within the region of interest of an examined tooth, and 3) measuring the variation of pixel values (or their derivatives, or variance, etc.) about a central measure within the region of interest to quantify the degree of damage to the enamel within the region of interest of an examined tooth and infer from that and other geometric measures, perhaps coupled with epidemiological studies, the likelihood that those observed lesions in addition to those correlated to the observed lesions, are likely to evolve into full blown caries.

By the method embodiment implemented in module 13, the calculated score is passed to program module 14, wherein the score is matched with a reference data score. The reference data score is itself obtained from reference image data 15 chosen from a library of reference image data and analyzed in the same manner as the acquired image data. The image data in the library are derived from epidemiological data that represent a predetermined probability range, or risk, that the directly observed (pre)lesion and those others possibly in the mouth but not directly observed represented in the reference image has of developing into a dental caries within a 6-18-month time frame. The matching of the score of the acquired data and that of the reference data results in a risk factor ranking, which is subsequently passed to the decision tree 16. Depending on the risk factor determined in module 14, the decision tree 16 determines which value to display to the practitioner-user. In one, three risk factors are associated with a range of probabilities that the prognosis of the lesion will develop into a cavity within a 6-18-month time period. As an example, the probability ranges can be assigned values of >83% for the highest risk factor, >71% for the intermediate risk factor, and <9% for the lowest risk factor, based on epidemiological evidence.

Some embodiments of the algorithm used to predict the prognosis of the pre-carious lesions can be segmented into several steps, the first step comprising imaging portions of the dentition in a manner that highlights the topographic distribution of pre-caries lesions as anticipated for QLF and other fluoroscopy based methods. That step could also or in addition comprise the assay of the integrated effect of the presence of pre-caries on the propagation through or scatter of incident energy from the tooth surface, as anticipated by ultrasound surface acoustic wave (SAW) analysis or optical tomography methods. A second step comprises extracting from the image, scatter or propagation data one or more of a variety of geometric factors characteristic of the subset of the entire patient's pre-caries lesion population that have contributed to the data. These geometric parameters can include, for example, the mean, median or mode of the surface area of the pre-caries lesions weighted by the brightness or darkness of those lesions relative to the mean, median or mode of the level of reflectivity, emissivity, attenuation, etc. of the entire tooth. A third step comprises a correlation, for example, of the results of a separate population-based study to the specific data extracted from the given patient of interest. This step could also incorporate a separate data stream such as patient age, socio-economic status, race, diet, etc. This epidemiological study of the third step would entail measuring the mathematical quantities of interest at a given time point from a group of test subjects and correlating those mathematical quantities to the subsequent evolution of the oral history of those test subjects.

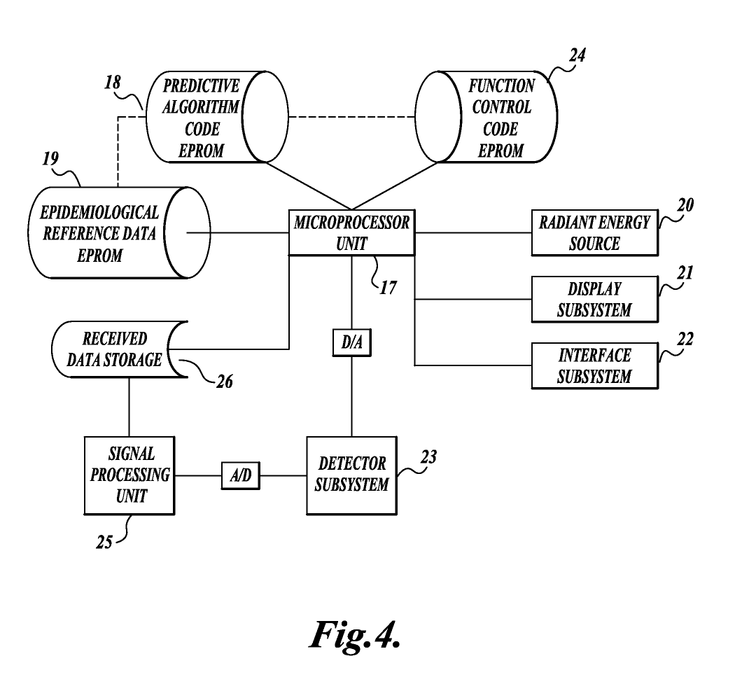

Referring now to FIG. 4, the block diagram shows how the predictive algorithm can be implemented, and how the electronic processing system controls all of the functions described above. Central microprocessor 17 is in communication with EPROM 18 which contains a manifestation of the predictive algorithm in the form of executable computer code. Epidemiological reference data is stored in a separate memory storage unit 19. The central processing unit also controls the functions of the radiant energy source 20, display subsystem 21, interface subsystem 22 and detector 23, via executable code contained in EPROM 24. Auxiliary subsystems such as a signal processing unit 25 aids in signal pre-conditioning before data is sent to storage unit 26. The dashed lines between the EPROM units indicate that they can be subdivisions or partitions of a single physical unit. Analog to digital (A/D) and digital to analog (D/A) functions are indicated in the figure as well.

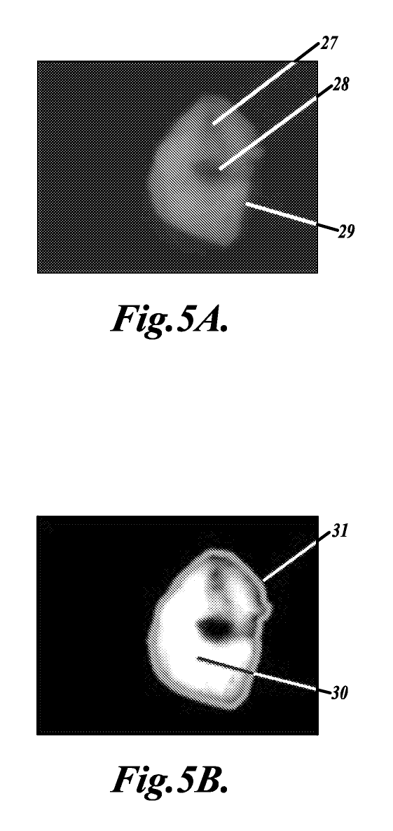

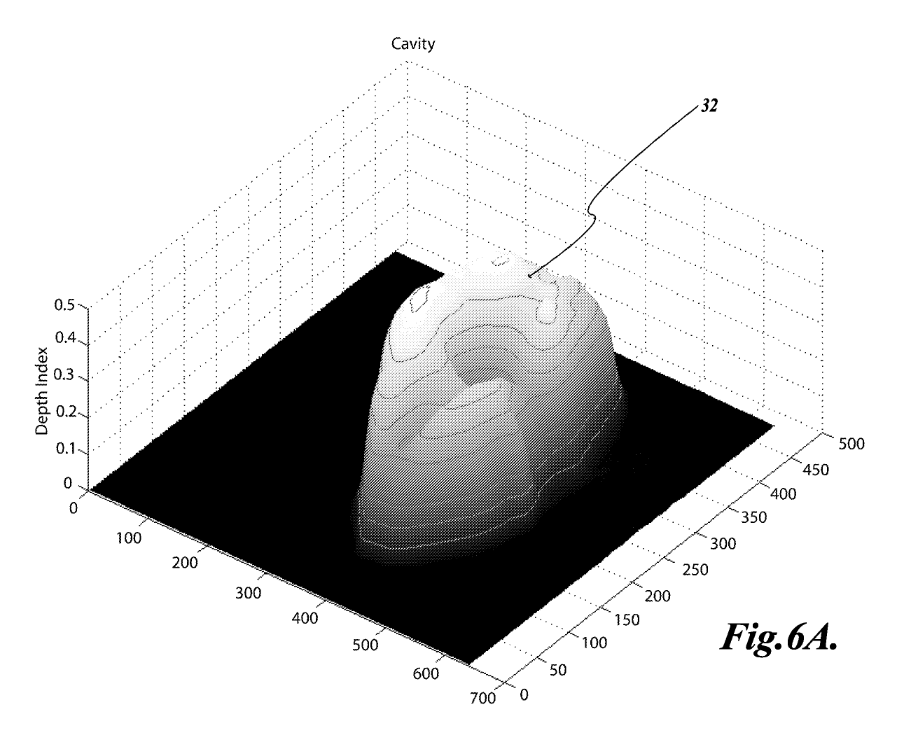

An example of QLF image data handling is now described. Referring to FIGS. 5a-b for purposes of illustration, tooth 27 with a known naturally occurring cavity 28 within a distribution of naturally occurring pre-caries lesions 29 is shown in FIG. 5a. Some filtering is done to the image to prepare it for analysis. RGB (red, green, blue) values are discarded from the image reducing it to a grayscale image. Image smoothing using preset filters is also preformed, which reduces the variability in brightness of the image. Before a tooth image can be analyzed for dental caries, a region of interest (ROI) must be defined for each tooth present in the image. A ROI is necessary because the algorithm correlates lower image intensity values (gray to dark) with lesion depth/severity, and areas surrounding a tooth may exhibit these lower intensity values, but must not be considered by the algorithm. The ROI is calculated by analyzing the distribution of intensity values in the image. A suitable cut-off value is chosen by analyzing the quantiles of this distribution, which distinguishes tooth from background levels. By sweeping across the image line by line, and working inward from the image edges, the ROI is constructed by locating the first occurrence of the cutoff value. An additional, imposed requirement of the ROI is that the image intensity values must be increasing towards the cutoff value along a given line, as this signifies the edge of a tooth. Such an ROI 30 is delineated by the outline 31 in FIG. 5b.

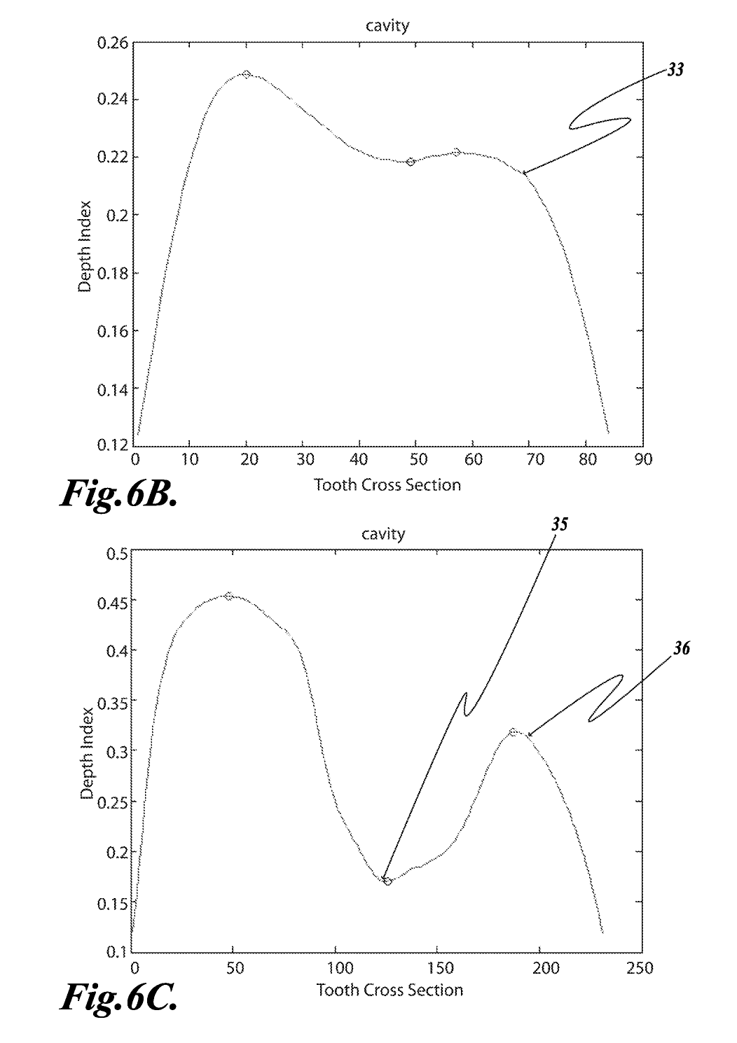

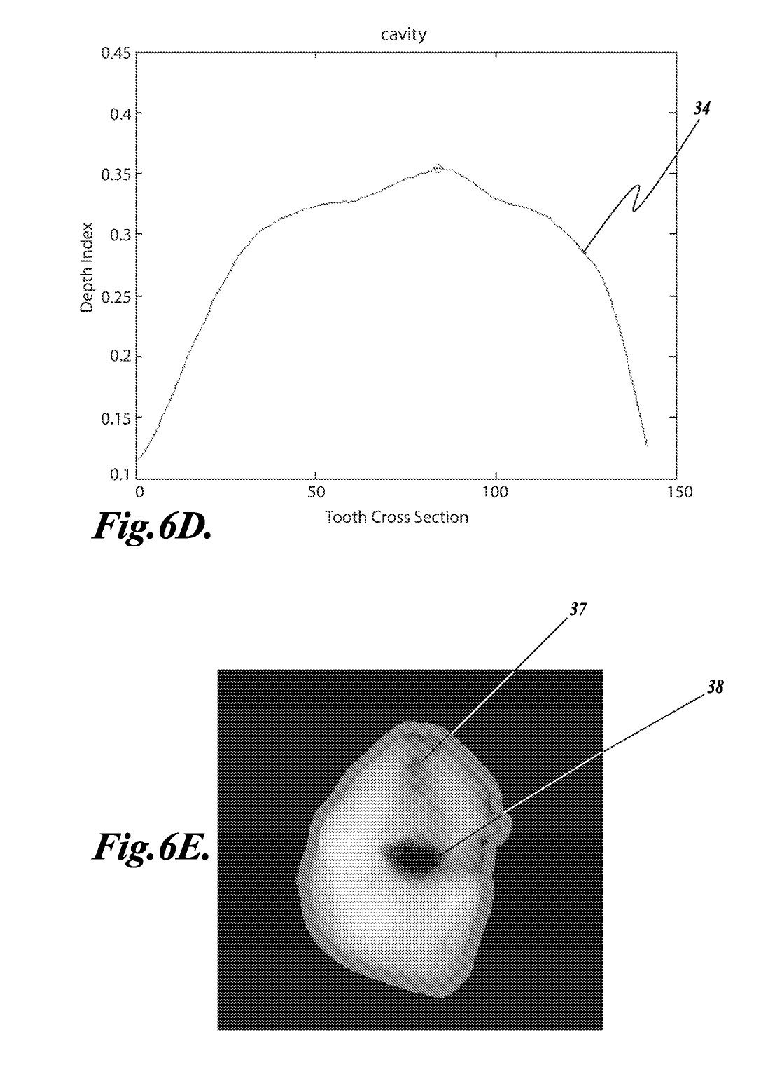

Once the ROI has been defined for a particular tooth, the algorithm can be put to work to determine the presence of dental caries or pre-caries lesions. Such (pre)caries are identified by a notable contrast between tooth brightness and (pre)caries lesion brightness. The process of translating pixel brightness values into geometrical data, such as a 3D rendering of the ROI, is now described. Sweeping across the ROI, line by line, a depth cross section is constructed by plotting the tooth width against a depth index. The depth index is a normalized index determined by dividing each image intensity value (on the given line) by the maximum brightness found on the tooth. FIGS. 6a-d illustrate this process. FIG. 6a shows a contour plot 32 constructed from the pixel values from the ROI 30 (FIG. 5b). FIGS. 5b-d show exemplary individual line scans which represent selected cross sections of the tooth in the ROI. The line scans 33 and 34 of FIGS. 6b and 6d, respectively, mark the two ends of the tooth that are lesion free and flank the section of the tooth containing pre-caries and the actual caries lesion. The lesions can be seen in FIG. 5c, where the pit 35 near the center of the scan is the actual caries lesion, and pre-caries lesion is indicated by the reduced height 36 of the tooth from deminerization on the right of the pit. The difference between the maximum and minimum values is then recorded for each scan line. (The tooth image will not be without some brightness variability due to lighting and its irregular surface). Thresholds are then defined that set the minimum depth and minimum width that the algorithm uses to determine if a contrast on the tooth is a lesion or not. This is more readily illustrated in FIG. 6e, which shows a false color image of the ROI wherein the caries lesion 28 is now represented as a black region near the center of the ROI, and the pre-caries lesions 29 are represented in grey in the figure.

After scanning the ROI line by line, the distribution of maximum depths is analyzed to make an assessment of the tooth. The 99% percentile of this distribution is used to represent the maximum depth present on the tooth. Thresholds for the severity of the lesion are hardcoded into the algorithm, and this maximum depth is used to make an assessment of the tooth. Concerning other methods of lesion detection (again, the first step in our approach), most of them at their core represent some sort of contrast analysis.

That is, they compare one region to another and then summarize that contrast with some metric. Then an assessment is made based on the collection of metrics obtained from the image. For the example algorithm presented here, maximum depth was chosen as the metric. Other valid choices could be dynamic range (max/min), depth standard deviation, depth quartiles (25%, 50%, 75%). The important point is that the anticipated device must first extract any of these mathematical descriptors from the data. Second, that device must have the ability to compare their values for a given patient to those from a population of test subjects whose own mathematical values, have, in turn, successfully correlated with the results of epidemiological assays of the oral health trajectory of those test subjects.

In a further embodiment, optical coherence tomography (OCT) is employed as the method of generating 3D images of pre-caries lesions. This methodology is gaining interest for dental diagnosis, particularly for detection of dental caries (see, e.g., U.S. Pat. No. 5,570,182). OCT is a low-coherence interferometric technique, wherein high resolution, cross-sectional tomographic images can be obtained using relatively simple optical componentry. In this technique, a single low-coherence broad-band white light or near infrared source is employed to supply both a signal and reference beam by use of a beam splitter. The signal beam is directed to a sample, while the reference beam is directed to a minor. Back-scattered light from the sample is then mixed with the reference beam at the face of the beam splitter, which acts now as an optical coupler, to create an interference pattern at a photodetector. The latter can be operated in the optical heterodyne detection mode to eliminate diffuse reflected light from the signal. The source wavelength region is chosen so that it can provide the greatest contrast between healthy enamel and carious enamel. In addition, it must be capable of penetration through the sample surface and probe sub-surface structure. Light in the particular wavelength range of 800-1200 nm is known to reflect more strongly from healthy enamel than from caries or pre-caries, as the latter is more absorbent in this wavelength region. Light in the 1200-1300 nm region can penetrate 3-4 mm into dental tissue. Light is reflected from sub-surface structural features such as interfaces between materials of even slightly varying refractive index, and strongly at abrupt interfaces, such in porous sections of the sub-surface enamel, and at the dentinoenamel junction. Because only coherent light is detected in this process, the very short coherence length (due to the polychromatic nature of the source light) of the recombined light returned to the sensor ensures that a very thin (axially) region of depth in the subsurface enamel is probed. The probing depth can be varied by simply varying the path length of the reference beam. By scanning the tightly focused signal beam laterally, high resolution (sub-micron in z and ca. 10-100 microns in x and y) 3D images can be built up from the data, revealing the penetration depth and lateral extent of a precarious lesion. Details of this method have been described in Fried et al., “Imaging Caries Lesions and Lesion Progression with Polarization Sensitive Optical Coherence Tomography” J. Biomed Opt., 2002 7: 618-27.

Example compact hand-held OCT dental imaging device can include those disclosed in U.S. Application No. 2008/0118886 (Liang et al.), incorporated herein by reference. Miniaturized mechanical scanning elements such as MEMS devices can be integrated with miniaturized optics to provide a compact OCT instrument. Techniques for providing high-speed scanning mechanisms can include those disclosed in U.S. Application No. 2009/0225324 (Bernstein et al.), incorporated herein by reference.

As another example of an alternative embodiment, Raman spectroscopy is used alone or in combination with OCT or QLF to further enhance the identification of pre-caries lesions with greater sensitivity. Dental applications of Raman spectroscopy have been developed in the 1990s and have been shown to be highly specific in determination of compositional and structural changes in tooth enamel, such as mineral orientation (polarized Raman spectroscopy) (in regards to Raman spectroscopy to dentistry, see H. Tsuda and J. Arends, “Raman spectroscopy in dental research: a short review of recent studies”, Adv. Dent Res. 1997 11: 539-47; M. T. Kirchner, H. G. M. Edwards, D. Lucy and A. M. Pollard, “Ancient and modern specimens of human teeth: a Fourier transform Raman spectroscopic study”, J. Raman Spectrosc. 1997 28: 171-178; S. Stewart et al., “Trends in early mineralization of murine calvarial osteoblastic cultures: a Raman microscopic study”, J. Raman Spectrosc. 2002 33: 536-543; A. Carden and M. D. Morris, “Application of vibrational spectroscopy to the study of mineralized tissues (review)”, J. Biomed. Opt 2000 5: 259-268; A. Carden et al. “Ultrastructural changes accompanying the mechanical deformation of bone tissue: A Raman imaging study”, Calcif. Tissue Int. 2003 72: 166-175; J. A. Timlin et al., “Raman spectroscopic imaging markers for fatigue-related microdamage in bovine bone”, Anal. Chem. 2000 72: 2229-2236; H. Ou-Yang et al. “Two-dimensional vibrational correlation spectroscopy of in vitro hydroxyapatite maturation”, Biopolymers 2000 57: 129-139; Y. Leung and M. D. Morris, “Characterization of the effects of postextraction treatments on human dentin-resin interface by micro-Raman spectroscopy”, J. Biomed. Opt. 1997 2: 120-124).

U.S. Application No. 2005/0283058 (Choo-smith et al.) discloses a combined Raman and OCT imaging device for incipient and mature dental caries detection. A good correlation exists between the OCT images and Raman spectral and imaging data for caries detection and characterization. It was shown that OCT imaging of lesion sites reveals deeper light penetration and stronger scattering, which is indicative of a highly porous structure. Simultaneously, Raman spectroscopic changes characteristic of enamel structural alterations were also observed to confirm demineralization. Demineralization is revealed by primarily monitoring changes in the phosphate P-O stretching and bending bands occurring at 490 cm⁻¹, 590 cm⁻¹ and 1045 cm⁻¹ in the hydroxyapatite makeup of the enamel. Changes in intensities of these bands result from structural and/or orientational changes of the hydroxyapatite microcrystallites, which normally are arranged in bundles to form rods or prisms and have a preferred orientation of the long axis perpendicular to the surface of the tooth. During demineralization, this preferred orientation can be randomized, giving rise to the spectral changes manifested in peak intensity variations (see H. Tsuda and J. Arends, “Orientational micro-Raman spectroscopy on hydroxyapatite single crystals and human enamel crystallites”, J. Dent. Res. 1994 73: 1703-1710; G. Leroy et al., “Human tooth enamel: a Raman polarized approach”, Appl. Spectrosc. 2002 56: 1030-1034). As further disclosed in U.S. Application No. 2005/0283058, this scrambling of the structural order in the enamel during demineralization can be more readily revealed using polarized Raman spectroscopy, wherein a polarizing filter is placed in the detection path for monitoring of parallel and perpendicular polarization with respect to the incident laser light polarization. Healthy enamel tissue is more anisotropic than carious tissue, thereby causing a greater difference between perpendicular and parallel spectra, whereas pre-carious and carious enamel where demineralization is taking place shows less variability between parallel and perpendicular polarized light. Polarization Raman data can be readily analyzed to reveal early onset of demineralization, hence detect pre-caries lesions. By careful analysis, Raman data can also be interpreted to reveal remineralized regions of enamel, revealing the auto-healing of pre-caries lesions. In regards to the present invention, the predictive algorithm disclosed herein predicts progression in remineralization, therefore, auto-healing, as well as demineralization of a pre-caries lesion; therefore the incorporation of Raman spectroscopy as a secondary analytical probe in the diagnostic device can be used to greatly enhance the prognostic power of the predictive algorithm of the present invention.

Strictly epidemiological methods exist for assaying the risk of a given patient for developing caries, based typically on age, diet, socio-economic status and the like. Other predictive methods assay the bacterial population and/or pH, for example, of saliva. None have proven sufficiently successful to guide patient care. One embodiment of the present invention, however, involves combining these ‘C.A.T.’ (Caries Assessment Tool) based approaches with pre-caries detection and prediction devices anticipated here.

In another embodiment, infrared backscattering or transluminescence can be employed as a method of caries detection and imaging. U.S. Application No. 2007/0134615 (Lovely), incorporated herein by reference, discloses a dental imaging system that uses infrared light between 800 and 1800 nm either transmitted through or scattered from the tooth under examination.

In another embodiment, the incorporation of an ultrasonic probe as a means of caries detection is disclosed. The potential of ultrasonic technology for the detection of dental caries, has been proposed in several instances. The sonic properties of the hard tissues of the tooth crown, in particular the outer tooth enamel, have been shown to be highly uniform among different teeth and individuals. Diagnostic ultrasonic echo profiles can be obtained from the enamel surface, and at the dentinoenamel and pulpodentinal junctions as well, using longitudinal ultrasonic irradiation (S. Y. Ng et al., Arch. Oral Biol. 1989 34:341-345). Changes in this profile have been described in instances of demineralization lesions indicating a substantial difference in the sonic conductivity between sound and dimineralized enamel (see S. Y. Ng et al., J. Dent. 1988 16:201-209 and WIPO Application No. WO 95/04506 (Hahn)). These changes are ascribed to conversion of intact enamel to demineralized enamel that has a higher water content than sound enamel. A family of patents issued to Bab et al., comprising U.S. Pat. Nos. 5,974,677, 6,162,177 and 6,190,318, parts of which have been incorporated herein by reference, disclose a surface wave ultrasonic diagnostic probe, wherein the device generates a surface ultrasonic wave that is transferred to a tooth under examination by the probe head. The surface waves travel along the smooth contours of the tooth, and are reflected by abrupt changes in surface morphology, such as a caries lesion or crown crack. These reflections are detected as distinctive echoes. Typically, the echo profiles comprise a primary echo and a secondary echo, the primary echo having a larger amplitude than the secondary echo, which can occur before and/or after the primary echo. Furthermore, the amplitude of reflected ultrasonic waves from lesions may be correlated with the depth of caries lesions. Amplitude data can be scored as radiolucency extent values, which, in turn, relate to the depth of a caries lesion (T. M. Marthaler Caries Res, 1970 4: 224-242). By categorizing echo amplitude data, caries can be distinguished also from other structural defects such as cracks in the enamel, and echoes from dentine and pulp regions of the tooth. Additionally, echoes from remineralized caries lesions can be distinguished from actively demineralized lesions by wave characteristics. These data can be input to the predictive algorithm of the present invention to determine the prognosis of an alleged pre-caries lesion.

From the foregoing it will be appreciated that although specific embodiments of the invention have been described herein for purposes of illustration, various modifications may be made without deviating from the spirit and scope of the invention. Accordingly, the invention is not limited except as by the appended claims.

What is claimed is:

1. A processor readable storage medium that includes data and instructions, wherein the execution of the instructions on a computing device provides for predicting the formation of dental caries in children and adults by enabling actions, comprising:
receiving intra-oral measurements of the surface and sub-surface structure of a subset of the teeth of an individual;
correlating the measurements with epidemiological data using a predictive algorithm; and
estimating a propensity of the patient toward developing dental caries within a set time frame based, at least in part, on the correlation of the measurements and the epidemiological data.

2. The processor readable storage medium of claim 1, wherein the time frame is in the range of 3 months to 18 months, and wherein the time range is based on the age of the patient.

3. The processor readable storage medium of claim 1, wherein the actions further comprise:
receiving data from a detection unit applied to a region of the patient's dentition;
analyzing the data to determine the properties of the pre-caries and/or caries lesions, wherein any observed lesion includes at least a subset of the total lesion population in the patient's dentition and any unobserved lesions include a remaining subset of the total lesion population in the patient's dentition; and
predicting the probability of development of caries in the patient's dentition by relating the analyzed data to epidemiological data.

4. The processor readable storage medium of claim 1, wherein receiving the measurements comprises receiving and recording radiant energy signals in the form of quantified light-induced fluorescence radiating from tooth enamel, coherent light resulting from a recombined reference beam, and a sample light beam backscattered from tooth enamel, backscattered Raman radiation, backscattered near infrared light, transluminescent near infrared light, surface acoustic waves from tooth enamel, or backscattered ultrasonic waves from tooth enamel of the patient's dentition obtained in vivo.

5. The processor readable storage medium of claim 1 using the predictive algorithm includes:
receiving data corresponding to a subset of a patient's teeth;
quantifying the received data corresponding to the individual's teeth; and
comparing the quantified data to reference data selected from a library of known reference data that is linked to an epidemiological probability of the population of pre-carious lesions directly observed and developing into dental caries within a set period of time for the patient.

6. The predictive algorithm according to claim 5, wherein the data is plurality of measurement values over portion of a patient's dentition partitioned into the elements of an array or a set of single values wherein each value is a measurement integrated over a region of a patient's dentition.

7. A system for predicting the formation of dental caries in children and adults comprising:
an instrument comprising a handle portion and a probe head portion, wherein the handle portion extents from the probe head portion, and a
a radiant energy source for irradiating the source energy incident on at least one tooth in a set of teeth in the mouth of an individual, the source being at least partially affixed to the probe head of the instrument;
a detector subsystem comprising the function of receiving a radiant energy signal emanating from a region of the enamel of the tooth in vivo, the detector subsystem being at least partially affixed to the probe head of the instrument and in proximity of the radiant energy source;
an electronic processing subsystem in communication with the radiant energy source and the detector subsystem, comprising the functions of controlling the radiant energy source and the detector subsystem, processing the signals received by the detector subsystem and predicting the probability of dental caries formation occurring in the patient's dentition within a set time period;
an display subsystem comprising the function of indicating to the user the probability of dental caries formation occurring in the patient's dentition within a set time period, wherein the display subsystem is in communication with the electronic processing subsystem;
an interface subsystem comprising the function of providing a communications link with an external computing device wherein the interface subsystem is in communication with the electronic processing subsystem.

8. The system of claim 7, wherein the radiant energy source is a broad-band visible light source, a broad-band near-infrared source, a near-infrared laser light source, a broad-band ultraviolet light source or an ultrasonic transducer.

9. The system of claim 7, wherein the radiant energy source comprises an array of LEDs, a scanning fiber optic element or any other arrangement of luminescent elements.

10. The system of claim 7, wherein the detection subsystem further comprises a CCD array element, at least one discrete photodiode element, an integrated photodiode array element, or an ultrasound transducing element.

11. The system of claim 7, wherein the electronic processing subsystem includes:
a microprocessor element;
a memory storage element containing instructions for the microprocessor, wherein the instructions correspond in part to the steps of the predictive algorithm;
a memory storage element containing the epidemiological reference data; and
a memory storage element containing the data received by the detector subsystem.

12. The system of claim 7, wherein the display subsystem includes a visual signaling device that is a plurality of colored indicators disposed on a portion of the implement substantially visible to the user wherein each indicator having a unique color signifying to a user a range of probabilities of caries formation within a set time period, a numerical readout disposed on a portion of the implement substantially visible to the user wherein the numerical value signifies to the user a range of probabilities of caries formation occurring in the patient's dention within a set time period, or a display disposed in a remote output device.

13. The system of claim 7, wherein the communications link is provided for wireless and wired connection with a computing device for uploading data to the computing device and is capable of data transmission rates allowing real time viewing of data on a computer display.
